(19) United States
(12) Patent Application Publication
(10) Pub. No.: US 2026/0117175 A1
(43) Pub. Date: Apr. 30, 2026

(54) In Vitro Culturing of Cells That Produce or Are Capable of Producing Antibodies

(71) Applicant: Emory University, Atlanta, GA (US)

(72) Inventors: Frances Eun-Hyung Lee, Atlanta, GA (US); Ignacio Sanz, Atlanta, GA (US); Doan C. Nguyen, Atlanta, GA (US)

(21) Appl. No.: 17/252,513

(22) PCT Filed: Jun. 17, 2019
(86) PCT No.: PCT/US2019/037551

§ 371 Date: Dec. 15, 2020

(51) Int. Cl. C12N 5/0781; C07K 16/00; C12N 5/00; C12Q 1/6869

(52) U.S. Cl. CPC C12N 5/0635; C07K 16/00; C12N 5/0081; C12Q 1/6869; C12N 2500/02; C12N 2500/32; C12N 2500/38; C12N 2500/84; C12N 2501/2306; C12N 2501/40

(57) ABSTRACT

This disclosure relates to growth media and environments for in vitro culturing of cells that produce or are capable of producing antibodies. In certain embodiments, the media comprises tyrosine 3-monooxygenase/tryptophan 5-monooxygenase activation protein zeta (YWHAZ), fibronectin, and optionally with other ingredients disclosed herein. In certain embodiments, the disclosure contemplates enclosures comprising culture compositions disclosed herein that are in ambient air or optionally in an environment wherein oxygen is absent or at a low concentration.

### CROSS-REFERENCE TO RELATED APPLICATIONS

This application claims the benefit of U.S. Provisional Application No. 62/685,889 filed Jun. 15, 2018. The entirety of this application is hereby incorporated by reference for all purposes.

### STATEMENT REGARDING FEDERALLY SPONSORED RESEARCH OR DEVELOPMENT

This invention was made with government support under AI121252 and AI078907 awarded by the National Institutes of Health. The government has certain rights in the invention.

### INCORPORATION-BY-REFERENCE OF MATERIAL SUBMITTED AS A TEXT FILE VIA THE OFFICE ELECTRONIC FILING SYSTEM (EFS-WEB)

The Sequence Listing associated with this application is provided in text format in lieu of a paper copy and is hereby incorporated by reference into the specification. The name of the text file containing the Sequence Listing is 18167PCT_ST25.txt. The text file is 19 KB, was created on Jun. 17, 2019, and is being submitted electronically via EFS-Web.

### BACKGROUND

Human long-lived plasma cells (LLPCs) are responsible for the long-term maintenance of protective serum antibodies. However, the maintenance of LLPCs remain poorly understood. Plasma cells readily die when removed from their in vivo surrounding. Thus, there is a need to identify in vitro methods of inducing the replication or prolonging the survival of cells capable of producing antibodies.

Roldan et al. report VLA-4-fibronectin interaction is required for the terminal differentiation of human bone marrow cells capable of spontaneous and high rate immunoglobulin secretion. J Exp Med. 1992, 175 (6): 1739-47.

Minges Wols et al. report the role of bone marrow-derived stromal cells in the maintenance of plasma cell longevity. See Journal of immunology, 2002, 169, 4213-4221.

Cassese et al. report plasma cell survival is mediated by synergistic effects of cytokines and adhesion-dependent signals. Journal of immunology, 2003, 171, 1684-1690.

Mesin et al. report Long-lived plasma cells from human small intestine biopsies secrete immunoglobulins for many weeks in vitro. J Immunol. 2011, 187(6): 2867-74.

Spencer et al. report the direct measurement of local oxygen concentration in the bone marrow of live animals. Nature. 2014, 508(7495): 269-73.

Hallily et al. report long-lived plasma cells are contained within the CD19(−) CD38(hi) CD138(+) subset in human bone marrow. Immunity, 2015, 43(1): 132-45.

WO 2016/201077 reports growth and survival compositions for cells capable of production antibodies.

U.S. patent application Ser. No. 15/992,174 reports methods of culturing and characterizing antibody secreting cells.

References cited herein are not an admission of prior art.

### SUMMARY

This disclosure relates to growth media and environments for in vitro culturing of cells that produce or are capable of producing antibodies. In certain embodiments, a cell culture composition comprises exogenously added 3-monooxygenase/tryptophan 5-monooxygenase activation protein zeta protein (YWHAZ) and exogenously added fibronectin, and optional in other exogenously added ingredients disclosed herein. In certain embodiments, the culture compositions comprise secretions of allogeneic mesenchymal stromal/stem cells in combination with the any of the ingredients disclosed herein.

In certain embodiments, a cell culture composition further comprises exogenously added A-proliferation-inducing ligand (APRIL). In certain embodiments, a cell culture composition further does or does not contain exogenously added IL-6. In certain embodiments, the cell culture composition cell culture composition is in an enclosure wherein the amount of oxygen is less than 5% by volume. In certain embodiments, the cell culture composition further comprises isolated cells capable of producing antibodies. In certain embodiments, the culture compositions further comprise an exogenously added components selected from buffering agent, amino acids, vitamins and a saccharide.

In certain embodiments, this disclosure relates to cell culture compositions comprising secretions of allogeneic mesenchymal stromal/stem cells and exogenously added tyrosine 3-monooxygenase/tryptophan 5-monooxygenase activation protein zeta protein (YWHAZ) and fibronectin, and optionally other ingredients disclosed herein. In certain embodiments, the cell culture composition further comprises exogenously added A-proliferation-inducing ligand (APRIL). In certain embodiments, cell culture composition is in an enclosure wherein the amount of oxygen is less than 5% by volume. In certain embodiments, the cell culture composition further comprises isolated cells capable of producing antibodies.

In certain embodiments, this disclosure relates to methods of sequencing a nucleic acid of antibodies that specifically bind to an antigen comprising isolating antibody secreting cells from a sample, providing separated single antibody secreting cells in a plurality of separate areas; contacting or mixing the separated single antibody secreting cells in the plurality of separated areas with a culture composition comprising secretions of allogeneic mesenchymal stromal/stem cells and exogenously added fibronectin and YWHAZ under conditions such that separated single antibody secreting cells replicate providing replicated homogenous antibody secreting cells in separate areas; identifying replicated homogenous antibody secreting cells that produce antibodies that specifically bind to an antigen; and sequencing a nucleic acid that encodes the antibody in the replicated homogenous antibody secreting cells that bind the antigen.

In certain embodiments, this disclosure relates to methods comprising isolating cells capable of producing antibodies produced in culture composition disclosed herein.

In certain embodiments, this disclosure relates methods of culturing cells that are capable of producing antibodies comprising mixing or contacting cells capable of producing antibody with a cell growth medium or culture composition disclosed herein.

In certain embodiments, cells that are capable of producing antibodies are plasma cells or antibody-secreting cells (ASCs).

In certain embodiments, the plasma cells have surface molecules in a pattern wherein no or low levels of CD19 are expressed, CD138 is expressed, and CD38 is expressed in higher levels than CD138.

In certain embodiments, culturing is done under conditions such that cells that are capable of producing antibodies secrete antibodies for more than or 10, 20, 30, 40, 50, 55, or 60 days.

In certain embodiments, this disclosure relates to composition comprising cells made by the process disclosed herein.

In certain embodiments, this disclosure relates to methods of sequencing a nucleic acid of antibodies that specifically bind to an antigen comprising isolating antibody secreting cells from a sample, providing separated single antibody secreting cells in a plurality of separate areas; mixing or contacting the separated single antibody secreting cells in the plurality of separated areas with a culture composition comprising exogenously added fibronectin and YWHAZ, and optionally other ingredients disclosed herein, under conditions such that separated single antibody secreting cells replicate providing replicated homogenous antibody secreting cells in separate areas; identifying replicated homogenous antibody secreting cells that produce antibodies that specifically bind to an antigen; and sequencing a nucleic acid that encodes the antibody in the replicated homogenous antibody secreting cells that bind the antigen. In certain embodiments, the culture composition further contains or does not contain exogenously added IL-6. In certain embodiments, the culture composition further comprises exogenously added A-proliferation-inducing ligand (APRIL), fragment, or variant thereof. In certain embodiments, the culture composition in an enclosure wherein the amount of oxygen is less than 5% by volume. In certain embodiments, the culture composition further comprises secretions of allogeneic mesenchymal stromal/stem cells.

In certain embodiments, the sample is blood, bone marrow, or product derived therefrom. In certain embodiments, the isolating antibody secreting cells is accomplished by fluorescence activated cell sorting. In certain embodiments, the antibody secreting cells have a cell surface profile of CD19(−), CD38(hi), and CD138(+). In certain embodiments, the antibody secreting cells have a cell surface profile of CD19+, CD27hi, and CD38hi. In certain embodiments, the sequencing is the variable region of the heavy chain of the antibody and/or the variable region of the light chain of the antibody produced by the homogenous antibody secreting cells.

In certain embodiments, this disclosure relates to methods of identifying cells that produce antibodies that bind to an antigen comprising isolating antibody secreting cells from a sample, providing isolated single antibody secreting cells; contacting or mixing the isolated antibody secreting cells with a culture composition comprising exogenously added fibronectin and YWHAZ, and optionally other ingredients disclosed herein, under conditions such that isolated antibody secreting cells replicate providing replicated antibody secreting cells; and identifying antibodies that specifically bind to the antigen being secreted from the cells indicating the presence of antigen binding antibody secreting cells in the sample. In certain embodiments, the antigen is a vaccine antigen or autoimmune antigen. In certain embodiments, the culture composition further contains or does not contain exogenously added IL-6. In certain embodiments, the culture composition further comprises exogenously added A-proliferation-inducing ligand (APRIL), fragment, or variant thereof. In certain embodiments, the culture composition in an enclosure wherein the amount of oxygen is less than 5% by volume. In certain embodiments, the culture composition further comprises secretions of allogeneic mesenchymal stromal/stem cells

In certain embodiments, this disclosure relates to methods of determining the effectiveness of a vaccination comprising administering a vaccine to a subject that results in the existence or production of a vaccine antigen in the subject; isolating antibody secreting cells from a sample, providing isolated antibody secreting cells contacting the isolated antibody secreting cells with a culture composition comprising exogenously added fibronectin and YWHAZ, and optionally other ingredients disclosed herein, under conditions such that isolated antibody secreting cells replicate providing replicated antibody secreting cells; and identifying replicated antibody secreting cells that produce antibodies that bind to the vaccine antigen. In certain embodiments, the time between administering the vaccine and isolating the antibody secreting cells from the sample is at least one year or two years or more. In certain embodiments, the culture composition further contains or does not contain exogenously added IL-6. In certain embodiments, the culture composition further comprises exogenously added A-proliferation-inducing ligand (APRIL), fragment, or variant thereof. In certain embodiments, the culture composition in an enclosure wherein the amount of oxygen is less than 5% by volume. In certain embodiments, the culture composition further comprises secretions of allogeneic mesenchymal stromal/stem cells.

In certain embodiments, this disclosure relates to methods of producing a hybridoma that produces antibodies that bind to an antigen comprising: isolating antibody secreting cells from a sample providing separated single antibody secreting cells in a plurality of separate areas; mixing or contacting the separated single antibody secreting cells with a culture composition comprising exogenously added fibronectin and YWHAZ, and optionally other ingredients disclosed herein, under conditions such that separated single cells replicate providing replicated homogenous antibody secreting cells; and identifying homogenous antibody secreting cells that produce antibodies that specifically bind to the antigen; and fusing an homogenous antibody secreting cell identified to produce antibodies that specifically bind the antigen with an immortalized cell providing hybridoma cells that produce antibodies that specifically bind to the antigen. In certain embodiments, the culture composition further contains or does not contain exogenously added IL-6. In certain embodiments, the culture composition further comprises exogenously added A-proliferation-inducing ligand (APRIL), fragment, or variant thereof. In certain embodiments, the culture composition in an enclosure wherein the amount of oxygen is less than 5% by volume. In certain embodiments, the culture composition further comprises secretions of allogeneic mesenchymal stromal/stem cells

In certain embodiments, this disclosure relates to methods of determining the efficacy of an autoimmune therapy comprising: administering an autoimmune drug to a subject diagnosed with an autoimmune disease; isolating antibody secreting cells from a sample of the subject, providing isolated antibody secreting cells; mixing or contacting the isolated antibody secreting cells with a culture composition comprising exogenously added fibronectin and YWHAZ, and optionally other ingredients disclosed herein, under conditions such that isolated antibody secreting cells replicate providing replicated antibody secreting cells; identifying replicated antibody secreting cells that produce auto-antibodies that specifically bind to an autoimmune antigen associated with the diagnosed autoimmune disease; quantifying an amount of auto-antibodies or cells that produce auto-antibodies in the sample; and correlating the amount to the efficacy of the autoimmune therapy. In certain embodiments, the culture composition further contains or does not contain exogenously added IL-6. In certain embodiments, the culture composition further comprises exogenously added A-proliferation-inducing ligand (APRIL), fragment, or variant thereof. In certain embodiments, the culture composition in an enclosure wherein the amount of oxygen is less than 5% by volume. In certain embodiments, the culture composition further comprises secretions of allogeneic mesenchymal stromal/stem cells

In certain embodiments, the culture composition comprises YWHAZ, IL-6, fibronectin, and APRIL and, typically, a saccharide, and optionally other ingredients disclosed herein. In certain embodiments, the disclosure contemplates cell culture compositions that further comprises components derived from proteins secreted from mesenchymal stromal/stem cells (MSCs). In certain embodiments, the disclosure contemplates enclosures comprising culture compositions disclosed herein that are in ambient air or optionally in an environment wherein oxygen is absent or at a low concentration.

In certain embodiments, the enclosure comprises ambient air or is optionally sealed from the atmosphere wherein the amount of oxygen is less than 10%, 5.0%, 2.5%, 2.0%, 1.5%, 1.0%, 0.5%, or 0.1% by volume.

In certain embodiments, the proteins secreted from mesenchymal stem cells are derived from extracting the proteins from a group of mesenchymal stromal/stem cells (MSCs) or are derived from replicating or non-replicating mesenchymal stromal/stem cells (MSCs) or irradiated mesenchymal stromal/stem cells (MSCs) in the growth medium. In certain embodiments, the mesenchymal stem cells are grown to near confluence and irradiated.

In certain embodiments, the culture compositions further comprises an exogenously added buffering agent, amino acids and vitamins. In certain embodiments, the culture compositions further comprises exogenously added blood. Typically, the blood is manipulated so that cells, platelets and/or clotting factor have been removed or are substantially absent, e.g., less than 5%, 3%, 2% or 1% by weight.

In certain embodiments, the disclosure relates to methods of culturing cells that are capable of producing antibodies comprising mixing cells capable of producing antibody with a cell growth medium disclosed herein. In certain embodiments, the cells that are capable of producing antibodies are plasma cells or antibody-secreting cells (ASCs). In certain embodiments, the plasma cells have surface molecules in a pattern wherein no or low levels of CD19 are expressed, CD138 is expressed, and CD38 is expressed in higher levels than CD138.

In certain embodiments, the culturing is done under conditions such that cells that are capable of producing and/or secreting antibodies survive or secret antibodies for more than 3, 4, 5, 6, 7, 8, 9, 10, 15, 20, 25, 30, 35, 40, 45, 50, 55, 56 or 60 days.

In certain embodiments, the disclosure relates to composition comprising cells made by the process disclosed herein.

In certain embodiments, the disclosure contemplates the use of or secretions of allogeneic mesenchymal stromal/stem cells (MSCs) or syngeneic mesenchymal stromal/stem cells (MSCs). In certain embodiments, the disclosure contemplates that the growth medium does not contain syngeneic mesenchymal stromal/stem cells (MSCs). In certain embodiments, the disclosure contemplates that the cells capable of producing and/or antibody secreting cells have no cell to cell contact with the allogeneic mesenchymal stromal/stem cells (MSCs) or syngeneic mesenchymal stromal/stem cells (MSCs). In certain embodiments, the disclosure contemplates that the survival media or culture compositions comprises a product derived from the secreted products of allogeneic mesenchymal stromal/stem cells (MSCs). In certain embodiments, allogeneic mesenchymal stromal/stem cells (MSCs) or syngeneic mesenchymal stromal/stem cells (MSCs) are bone marrow derived mesenchymal stromal/stem cells (MSCs).

In certain embodiments, any of the composition or methods disclosed above or herein may contain one or more of the following as exogenously added components identified from the genomic approach described herein. In certain embodiments, these ingredients/components include: Beta-actin (ACTB), Heterogeneous nuclear ribonucleoprotein A1 (HNRNPA1), Heat shock 70 kDa protein 8 (HSPA8), heat shock 70 kDa protein 5 (HSPA5), Tubulin beta chain (TUBB), alpha-enolase (ENO1), Vimentin (VIM), Eukaryotic elongation factor 2 (EEF2), heat shock protein family D (Hsp60) member 1 (HSPD1), Glyceraldehyde 3-phosphate dehydrogenase (GAPDH), or combinations thereof.

### BRIEF DESCRIPTION OF THE FIGURES

FIG. 1A shows data indicating BM-MSC support in vitro survival of blood ASC. Short-term survival of blood ASC in irradiated MSC (iMSC) co-cultures. FACS purified blood ASC (1000/well) were cultured in RPMI with 10% FBS (R10) (No MSC) or co-cultured with 50,000 BMMSC (MSC) for 7 days (p<0.001). Representative images of Elispot wells are shown. Elispot assays were performed on the indicated day and the frequency (%) of IgG-secreting ASC were calculated based on the maximal Elispots.

FIG. 1B shows data on long-term survival of blood ASC and iMSC co-cultures from two different adult blood samples after vaccination. Five hundred to 1,500 ASC with 25,000 or 30,000 iMSC were co-cultured in each well and IgG Elispots were performed on designated days.

FIG. 1C shows data on prosurvival support of BM-MSC is independent of cell-cell contact. Same number of blood ASC were cultured in R10 (Media), co-cultured with 50,000 iMSC (iMSC), in transwells with ASC and 50,000 iMSC in separate chambers (TW), or iMSC secretome (Secretome).

FIG. 1D shows representative Elispot wells.

FIG. 1E shows data on long-term survival of blood ASC in MSC secretome. One thousand ASC were cultured in MSC secretome or with media alone.

FIG. 2A shows data indicating MSC secretome restores the secretory function of sorted ASC. Flow cytometric analysis of intracellular Ki-67 expression of blood ASC.

FIG. 2B shows data on the effects of antibody staining and sorting on ASC survival and function. IgG Elispot assays were performed on the same number of PBMC that were untouched (No Stain or Sort), only stained with antibodies and not FAC sorted (Stain), only FAC sorted and not stained with antibodies (Sort), or both stained and FAC sorted (Stain & Sort). Maximal number of IgG Elispots occurred in the No Stain or Sort wells and normalized to 100%.

FIG. 2C shows data on BrdU incorporation of MSC and ASC. BrdU was added to ASC cultured in the secretome or secretome alone. Triplicate cultures of dividing noniMSC alone with and without BrdU served as positive and negative controls. BrdU was also added to conditions of secretome alone or secretome with ASC.

FIG. 2D shows data on no BM-derived ASC contamination in iMSC co-cultures or MSC secretome. IgG Elispot assays were performed on iMSC (iMSC) or MSC secretome (Secretome). Blood ASC in MSC secretome cultures (ASC+Secretome) served as positive control.

FIG. 3A shows data indicating APRIL together with the MSC secretome enhances blood ASC survival. Blood ASC survival is enhanced by the MSC secretome with exogenous APRIL. First two rows: sorted blood ASC were cultured in R10 alone or in media with exogenous APRIL for 7 days. Second two rows: blood ASC were cultured in MSC secretome alone or the secretome with APRIL. Blood ASC immediately from FAC sorting are shown (day 0). Representative images of Elispot wells are shown.

FIG. 3B shows a graphic plot of % survival of IgG ASC in media alone (R10) (open circles), APRIL alone (open squares), secretome alone (solid circles), or secretome+April (triangles) (p<10-6, between secretome alone & secretome+April).

FIG. 3C shows data on exogenous APRIL together with the MSC secretome enhances ASC survival in short-term cultures. Blood ASC from 4 different subjects after vaccination were cultured in media alone (open circles), secretome alone (solid circles), or secretome+April (triangles). Percentage of IgG Elipsots normalized to maximal frequency on days 1-3. Shown are p-values between secretome alone & secretome+April.

FIG. 3D shows exogenous APRIL with the MSC secretome enhances ASC survival in long-term cultures. ASC cultured with the MSC secretome (solid circles) or MSC secretome+April (triangles).

FIG. 3E shows data on the treatment of MSC secretome with anti-IL-6 antibodies (anti-IL-6) diminishes blood ASC survival. Blood ASC were cultured in MSC secretome or MSC secretome treated with anti-IL-6 for 1, 3, 7 days and IgG Elispots were performed, between secretome vs secretome+anti-IL-6). The frequency of isotype controls were similar to MSC secretome.

FIG. 4A is a flow diagram on integrated bioinformatics of MSC secretome proteomics with transcriptomics of blood ASC and BM LLPC.

FIG. 4B shows proteomics of distinct MSC secretome fractions. Three fractions strongly support blood ASC survival: iMSC secretome (filled square; left and right panels), noniMSC secretome (open triangles; left panel), and supernatant fractionated by ultracentrifugation of iMSC secretome (open circles; left panel); and three fractions with decreased ASC survival: conventional media (R10) (open circles; left panel), supernatant fractionated by overnight ultracentrifugation of noniMSC secretome (black open triangles; left panel), and secretome from blood (not BM) adherent cells (black open triangles; right panel).

FIG. 4C shows a heat map of the 2,558 DEG between 17 blood ASC (PB ASC) obtained from 7 healthy subjects and BM LLPC (LLPC) obtained from 4 adult subjects.

FIG. 4D shows HIPPIE analysis of 91 MSC protein revealed 4,429 potential protein partners (PPI). Overlap of the 4,429 PPI with the 2,558 DEG uncovered 556 overlapping gene/protein targets and led to 20 statistically significant GSEA hallmark pathways.

FIG. 4E provides of the aforementioned 91 MSC secretome proteins, FN-1 and YWHAZ had the highest number (118 and 119, respectively) of potential interacting partners. Of these potential partners, 31 were shared between both FN-1 and YWHAZ. From the 20 GSEA pathways, FN-1 and YWHAZ were found to be involved in these 10 potential GSEA hallmark pathways.

FIG. 5A shows data on the treatment of MSC secretome with antibodies targeting FN-1 diminishes blood ASC survival. Blood ASC were cultured in MSC secretome alone (white bar) or MSC secretome treated with antibodies targeting FN-1 or appropriate isotype controls (gray bar).

FIG. 5B shows data for antibodies targeting YWHAZ.

FIG. 5C shows sensitivity and resistance to Rapamycin in blood ASC. Blood ASC from 3 healthy subjects or BM LLPC from 3 adults were sorted and cultured in untreated MSC secretome (black bar) or MSC secretome treated with rapamycin (gray bar). The frequency of vehicle controls (DMSO) was comparable to that of untreated MSC secretome.

FIG. 5D shows data in BM LLPC.

FIG. 6A show data indicating survival of blood ASC is enhanced under hypoxic conditions with the MSC secretome and APRIL. Circulating ASC post-tetanus vaccination from one healthy adult were cultured under the following conditions in normoxia: media alone, secretome alone or secretome+April and under hypoxia conditions: media alone, secretome alone, or secretome+April. Percentage of IgG Elispots were normalized to the maximal frequency on day 3. Data is representative of 4 different blood ASC from 4 adults after vaccination.

FIG. 6B shows representative images of Elispot wells from the experiments in 6A are shown. Number of ASC per well: 1,333 except on wells from days 21-56 for MSC secretome+April in normoxia or hypoxia to show images in a countable range.

FIG. 7A shows a schematic for making a human cell-free in vitro BM mimetic (i.e. MSC secretome).

FIG. 7B shows data indicating FN1, HSPD1, GAPDH, and hnRNPA1 are involved in the in vitro survival of blood ASC. Data represents the Ig secretion of blood ASC using a cell-free in vitro BM mimic in the presence of specific Abs, e.g., anti-YWHAZ (aka 14-3-3) revealing FN1, YWHAZ, GAPDH, HSPD1, and hnRNPA1 as the protein factors important for the in vitro ASC/PC survival.

FIG. 7C shows data on the Ig secretion of BM PC using a cell-free in vitro BM mimic in the presence of specific Abs.

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise.

The term “mesenchymal stromal cells” refers to the subpopulation of fibroblast or fibroblast-like nonhematopoietic cells with properties of plastic adherence and capable of in vitro differentiation into cells of mesodermal origin which may be derived from bone marrow, adipose tissue, umbilical cord (Wharton's jelly), umbilical cord perivascular cells, umbilical cord blood, amniotic fluid, placenta, skin, dental pulp, breast milk, and synovial membrane, e.g., fibroblasts or fibroblast-like cells with a clonogenic capacity that can differentiate into several cells of mesodermal origin, such as adipocytes, osteoblasts, chondrocytes, skeletal myocytes, or visceral stromal cells. The term, “mesenchymal stem cells” refers to the cultured (self-renewed) progeny of primary mesenchymal stromal cell populations. Mesenchymal stromal/stem cells (MSCs) refers to mesenchymal stromal and/or mesenchymal stem cells.

Bone marrow derived mesenchymal stromal cells are typically expanded ex vivo from bone marrow aspirates to confluence. Certain mesenchymal stromal/stem cells (MSCs) share a similar set of core markers and properties. Certain mesenchymal stromal/stem cells (MSCs) may be defined as positive for CD105, CD73, and CD90 and negative for CD45, CD34, CD14 or CD11b, CD79α or CD19, and HLA-DR surface markers, and have the ability to adhere to plastic. See Dominici et al. Minimal criteria for defining multipotent mesenchymal stromal cells. The International Society for Cellular Therapy position statement. Cytotherapy. 2006, 8 (4): 315-7.

Plasma cells (PCs) in human BM express high amounts of CD38. Long lived plasma cells can be obtained from human bone marrow cells, e.g., from iliac crest aspirates. Cells may be separated by flow cytometry. One can us FACS to remove lymphocytes having CD3 or CD14 expression (non-T cells, non-monocytes) and IgD cells (to eliminate late transitional and naive B cells). The remaining cells may be divided remove CD19 cell populations and subsequently obtained by the expression of both CD138 and CD38. In certain embodiments, antibody secreting cells such as ACSs or PC provide immunoglobulin secretion of at or more than 100, 125, 150 or 167±23 pg/cell/day.

The term “fluorescence-activated cell sorting” or “FACS” refers to a method of sorting a mixture of cells into two or more areas, typically one cell at a time, based upon the fluorescent characteristics of each cell. It is typically accomplished by applying an electrical charge and separating by movement through an electrostatic field. Fluorescent antibodies with epitopes to cell surface markers can be mixed with cells to mark the cells or cells can be transfected with fluorescent probes or molecular beacons that bind to mRNA. Typically, in FACS, a vibrating mechanism causes a stream of cells to break into individual droplets. Just prior to droplet formation, cells in a fluid pass through an area for measuring fluorescence of the cell. An electrical charging mechanism is configured at the point where the stream breaks into droplets. Based on the fluorescence intensity measurement, a respective electrical charge is imposed on the droplet as it breaks from the stream. The charged droplets then move through an electrostatic deflection system that diverts droplets into areas based upon their relative charge. In some systems, the charge is applied directly to the stream, and the droplet breaking off retains charge of the same sign as the stream. In other systems, a charge is provided on a conduit inducing an opposite charge on the droplet.

“YWHAZ” refers to tyrosine 3-monooxygenase/tryptophan 5-monooxygenase activation protein zeta protein, also referred to as 14-3-3 protein zeta (14-3-3ζ) protein. NCBI Reference Sequence: NP_001129171.1. The human protein is encoded by the YWHAZ gene on chromosome 8. 14-3-3 proteins generally form homo- or heterodimers. In certain embodiments, a growth medium disclosed herein comprises exogenously added YWHAZ, fragment, or variant thereof. In certain embodiments, the variant has greater than 30, 40, 50, 60, 70, 80, 85, 90, 95, 96, 97, 98 or 99% identity or similarity to (SEQ ID NO: 4) MDKNELVQKAKLAEQAERYDDMAACMKSVTEQGAELSNEERNLLSVAYKNVVGARR SSWRVVSSIEQKTEGAEKKQQMAREYREKIETELRDICNDVLSLLEKFLIPNASQAESKV FYLKMKGDYYRYLAEVAAGDDKKGIVDQSQQAYQEAFEISKKEMQPTHPIRLGLALNF SVFYYEILNSPEKACSLAKTAFDEAIAELDTLSEESYKDSTLIMQLLRDNLTLWTSDTQG DEAEAGEGGEN.

In certain embodiments, a growth medium disclosed herein comprises exogenously added YWHAZ, fragment, or variant thereof. In certain embodiments, YWHAZ is in the growth medium at a concentration of greater than 0.001×10⁻³%, 0.002×10⁻³%, 0.003×10⁻³%, 0.004×10⁻³%, 0.005×10⁻³%, 0.007×10⁻³%, 0.010×10⁻³%, 0.020×10⁻³%, 0.030×10⁻³%, 0.050×10⁻³%, 0.10×10-3%, 0.20×10⁻³%, 0.30×10⁻³%, 0.50×10⁻³%, 1.0×10⁻³%, 1.5×10⁻³%, 2.0×10⁻³%, 0.001%, 0.002%, 0.003%, 0.004%, 0.005%, 0.007%, 0.010%, 0.020%, 0.030%, 0.050%, 0.10%, 0.20%, 0.30%, 0.50%, 1.0%, 1.5%, 2.0%, by weight.

“Fibronectin” refers to either plasma insoluble fibronectin typically produced by fibroblasts, e.g., in an extracellular matrix, and plasma soluble fibronectin produced in the liver by hepatocytes sometimes referred to as “cold-insoluble globulin” which is a protein component of blood plasma. Fibronectin exists as a protein dimer, consisting of two monomers linked near the C-terminus by a pair of disulfide bonds. A typical fibronectin contains 12 type I modules, 2 type II modules, and 15-17 type III modules. The number of modules varies based on alternative gene splicing. There are two alternatively spliced segments in fibronectin due to alternative exon usage: extra domain A (EDA) located between the 11th and 12th of type III modules, and extra domain B (EDB) between the seventh and eighth type III modules. Plasma fibronectin typically lacks EDA and EDB sequences.

In certain embodiments, a growth medium disclosed herein comprises exogenously added fibronectin, fragment, or variant thereof. In certain embodiments, fibronectin is in the growth medium at a concentration of greater than 0.001×10⁻³%, 0.002×10⁻³%, 0.003×10⁻³%, 0.004×10⁻³%, 0.005×10⁻³%, 0.007×10⁻³%, 0.010×10⁻³%, 0.020×10⁻³%, 0.030×10⁻³%, 0.050×10⁻³%, 0.10×10⁻³%, 0.20×10⁻³%, 0.30×10⁻³%, 0.50×10⁻³%, 1.0×10⁻³%, 1.5×10⁻³%, 2.0×10⁻³%, 0.001%, 0.002%, 0.003%, 0.004%, 0.005%, 0.007%, 0.010%, 0.020%, 0.030%, 0.050%, 0.10%, 0.20%, 0.30%, 0.50%, 1.0%, 1.5%, 2.0%, by weight.

The protein “APRIL” refers to tumor necrosis factor superfamily member 13, which is a ligand for B-cell maturation antigen, a member of the tumor necrosis factor (TNF) receptor family. In certain embodiments, a growth medium disclosed herein comprises exogenously added APRIL, fragment, or variant thereof. In certain embodiments, the variant has greater than 30, 40, 50, 60, 70, 80, 85, 90, 95, 96, 97, 98 or 99% identity or similarity to (SEQ ID NO: 1) MPASSPFLLAPKGPPGNMGGPVREPALSVALWLSWGAALGAVACAMALLTQQTELQS LRREVSRLQGTGGPSQNGEGYPWQSLPEQSSDALEAWENGERSRKRRAVLTQKQKKQ HSVLHLVPINATSKDDSDVTEVMWQPALRRGRGLQAQGYGVRIQDAGVYLLYSQVLFQ DVTFTMGQVVSREGQGRQETLFRCIRSMPSHPDRAYNSCYSAGVFHLHQGDILSVIIPRA RAKLNLSPHGTFLGFVKL.

In certain embodiments, APRIL is in the growth medium at a concentration of greater than 0.001×10⁻³%, 0.002×10⁻³%, 0.003×10⁻³%, 0.004×10⁻³%, 0.005×10⁻³%, 0.007×10⁻³%, 0.010×10⁻³%, 0.020×10⁻³%, 0.030×10⁻³%, 0.050×10⁻³%, 0.10×10⁻³%, 0.20×10⁻³%, 0.30×10⁻³%, 0.50×10⁻³%, 1.0×10⁻³%, 1.5×10⁻³%, 2.0×10⁻³%, 0.001%, 0.002%, 0.003%, 0.004%, 0.005%, 0.007%, 0.010%, 0.020%, 0.030%, 0.050%, 0.10%, 0.20%, 0.30%, 0.50%, 1.0%, 1.5%, 2.0%, by weight.

In response to injury, inflammatory cells such as neutrophil granulocytes and macrophages secrete a number of cytokines, most notable of which are the interleukins IL-1, IL-6 and IL-8, and TNFα. “IL-6” refers to the Interleukin-6 protein. IL-6 signals through a cell-surface type I cytokine receptor complex consisting of the ligand-binding IL-6Rα chain (CD126), and the signal-transducing component gp130 (CD130). IL-6 is thought to be involved in the activation of the immune system, regenerative processes, and regulation of metabolism.

In certain embodiments, a growth medium disclosed herein comprises exogenously added IL-6, fragment, or variant thereof. In certain embodiments, the variant has greater than 30, 40, 50, 60, 70, 80, 85, 90, 95, 96, 97, 98 or 99% identity or similarity to isoform 1 (SEQ ID NO: 2) MNSFSTSAFGPVAFSLGLLLVLPAAFPAPVPPGEDSKDVAAPHRQPLTSSERIDKQIRYIL DGISALRKETCNKSNMCESSKEALAENNLNLPKMAEKDGCFQSGFNEETCL VKIITGLLE FEVYLEYLQNRFESSEEQARAVQMSTKVLIQFLQKKAKNLDAITTPDPTTNASLLTKLQA QNQWLQDMTTHLILRSFKEFLQSSLRALRQM.

In certain embodiments, the variant has greater than 30, 40, 50, 60, 70, 80, 85, 90, 95, 96, 97, 98 or 99% identity or similarity to isoform 2 (SEQ ID NO: 3), CESSKEALAENNLNLPKMAEKDGCFQSGFNEETCLVKIITGLLEFEVYLEYLQNRFESSE EQARAVQMSTKVLIQFLQKKAKNLDAITTPDPTTNASLLTKLQAQNQWLQDMTTHLIL RSFKEFLQSSLRALRQM.

In certain embodiments, IL-6 is in the growth medium at a concentration of greater than 0.001×10⁻³%, 0.002×10⁻³%, 0.003×10⁻³%, 0.004×10⁻³%, 0.005×10⁻³%, 0.007×10⁻³%, 0.010×10⁻³%, 0.020×10⁻³%, 0.030×10⁻³%, 0.050×10⁻³%, 0.10×10⁻³%, 0.20×10⁻³%, 0.30×10⁻³%, 0.50×10⁻³%, 1.0×10⁻³%, 1.5×10⁻³%, 2.0×10⁻³%, 0.001%, 0.002%, 0.003%, 0.004%, 0.005%, 0.007%, 0.010%, 0.020%, 0.030%, 0.050%, 0.10%, 0.20%, 0.30%, 0.50%, 1.0%, 1.5%, 2.0%, by weight.

The protein “HSPD1” refers to 60 kDa heat shock protein. The human reference protein sequence is provided in NCBI Reference Sequence: NP_002147.2. In certain embodiments, a growth medium disclosed herein comprises exogenously added HSPD1, fragment, or variant thereof. In certain embodiments, the variant has greater than 30, 40, 50, 60, 70, 80, 85, 90, 95, 96, 97, 98 or 99% identity or similarity (SEQ ID to NO: 5) MLRLPTVFRQMRPVSRVLAPHLTRAYAKDVKFGADARALMLQGVDLLADAVAVTMG PKGRTVIIEQSWGSPKVTKDGVTVAKSIDLKDKYKNIGAKLVQDVANNTNEEAGDGTT TATVLARSIAKEGFEKISKGANPVEIRRGVMLAVDAVIAELKKQSKPVTTPEEIAQVATIS ANGDKEIGNIISDAMKKVGRKGVITVKDGKTLNDELEIIEGMKFDRGYISPYFINTSKGQ KCEFQDAYVLLSEKKISSIQSIVPALEIANAHRKPLVIIAEDVDGEALSTL VLNRLKVGLQ VVAVKAPGFGDNRKNQLKDMAIATGGAVFGEEGLTLNLEDVQPHDLGKVGEVIVTKD DAMLLKGKGDKAQIEKRIQEIIEQLDVTTSEYEKEKLNERLAKLSDGVAVLKVGGTSDV EVNEKKDRVTDALNATRAAVEEGIVLGGGCALLRCIPALDSLTPANEDQKIGIEIIKRTLK IPAMTIAKNAGVEGSLIVEKIMQSSSEVGYDAMAGDFVNMVEKGIIDPTKVVRTALLDA AGVASLLTTAEVVVTEIPKEEKDPGMGAMGGMGGGMGGGMF.

In certain embodiments, HSPD1 is in the growth medium at a concentration of greater than 0.001×10⁻³%, 0.002×10⁻³%, 0.003×10⁻³%, 0.004×10⁻³%, 0.005×10⁻³%, 0.007×10⁻³%, 0.010×10⁻³%, 0.020×10⁻³%, 0.030×10⁻³%, 0.050×10⁻³%, 0.10×10⁻³%, 0.20×10⁻³%, 0.30×10⁻³%, 0.50×10⁻³%, 1.0×10⁻³%, 1.5×10⁻³%, 2.0×10⁻³%, 0.001%, 0.002%, 0.003%, 0.004%, 0.005%, 0.007%, 0.010%, 0.020%, 0.030%, 0.050%, 0.10%, 0.20%, 0.30%, 0.50%, 1.0%, 1.5%, 2.0%, by weight.

The protein “GAPDH” refers to glyceraldehyde-3-phosphate dehydrogenase. The human reference protein sequence is provided in NCBI Reference Sequence: NP_001276674.1 (isoform 1) and NCBI Reference Sequence: NP_001243728.1 (isoform 1). In certain embodiments, a growth medium disclosed herein comprises exogenously added GAPDH, fragment, or variant thereof. In certain embodiments, the variant has greater than 30, 40, 50, 60, 70, 80, 85, 90, 95, 96, 97, 98 or 99% identity or similarity to (SEQ ID NO: 6) MVYMFQYDSTHGKFHGTVKAENGKL VINGNPITIFQERDPSKIKWGDAGAEYVVESTG VFTTMEKAGAHLOGGAKRVIISAPSADAPMFVMGVNHEKYDNSLKIISNASCTTNCLAP LAKVIHDNFGIVEGLMTTVHAITATQKTVDGPSGKLWRDGRGALQNIIPASTGAAKAVG KVIPELNGKLTGMAFRVPTANVSVVDLTCRLEKPAKYDDIKKVVKQASEGPLKGILGYT EHQVVSSDFNSDTHSSTFDAGAGIALNDHFVKLISWYDNEFGYSNRVVDLMAHMASKE.

In certain embodiments, GAPDH is in the growth medium at a concentration of greater than 0.001×10⁻³%, 0.002×10⁻³%, 0.003×10⁻³%, 0.004×10⁻³%, 0.005×10⁻³%, 0.007×10⁻³%, 0.010×10⁻³%, 0.020×10⁻³%, 0.030×10⁻³%, 0.050×10⁻³%, 0.10×10⁻³%, 0.20×10⁻³%, 0.30×10⁻³%, 0.50×10⁻³%, 1.0×10⁻³%, 1.5×10⁻³%, 2.0×10⁻³%, 0.001%, 0.002%, 0.003%, 0.004%, 0.005%, 0.007%, 0.010%, 0.020%, 0.030%, 0.050%, 0.10%, 0.20%, 0.30%, 0.50%, 1.0%, 1.5%, 2.0%, by weight.

The protein “HNRNPA1” refers to heterogeneous nuclear ribonucleoprotein A1. The human reference protein sequence is provided in NCBI Reference Sequence: NP_002127.1 (isoform a) and NCBI Reference Sequence: NP_112420.1 (isoform b). In certain embodiments, a growth medium disclosed herein comprises exogenously added HNRNPA1, fragment, or variant thereof. In certain embodiments, the variant has greater than 30, 40, 50, 60, 70, 80, 85, 90, 95, 96, 97, 98 or 99% identity or similarity to (SEQ ID NO: 7) MSKSESPKEPEQLRKLFIGGLSFETTDESLRSHFEQWGTLTDCVVMRDPNTKRSRGFGFV TYATVEEVDAAMNARPHKVDGRVVEPKRAVSREDSQRPGAHLTVKKIFVGGIKEDTEE HHLRDYFEQYGKIEVIEIMTDRGSGKKRGFAFVTFDDHDSVDKIVIQKYHTVNGHNCEV RKALSKQEMASASSSQRGRSGSGNFGGGRGGGFGGNDNFGRGGNFSGRGGFGGSRGG GGYGGSGDGYNGFGNDGSNFGGGGSYNDFGNYNNQSSNFGPMKGGNFGGRSSGPYGG GGQYFAKPRNQGGYGGSSSSSSYGSGRRF.

In certain embodiments, HNRNPA1 is in the growth medium at a concentration of greater than 0.001×10⁻³%, 0.002×10⁻³%, 0.003×10⁻³%, 0.004×10⁻³%, 0.005×10⁻³%, 0.007×10⁻³%, 0.010×10⁻³%, 0.020×10⁻³%, 0.030×10⁻³%, 0.050×10⁻³%, 0.10×10⁻³%, 0.20×10⁻³%, 0.30×10⁻³%, 0.50×10⁻³%, 1.0×10⁻³%, 1.5×10⁻³%, 2.0×10⁻³%, 0.001%, 0.002%, 0.003%, 0.004%, 0.005%, 0.007%, 0.010%, 0.020%, 0.030%, 0.050%, 0.10%, 0.20%, 0.30%, 0.50%, 1.0%, 1.5%, 2.0%, by weight.

Sequence “identity” refers to the number of exactly matching amino acids (expressed as a percentage) in a sequence alignment between two sequences of the alignment calculated using the number of identical positions divided by the greater of the shortest sequence or the number of equivalent positions excluding overhangs wherein internal gaps are counted as an equivalent position. For example, the polypeptides GGGGGG (SEQ ID NO: 8) and GGGGT (SEQ ID NO: 9) have a sequence identity of 4 out of 5 or 80%. For example, the polypeptides GGGPPP (SEQ ID NO: 10) and GGGAPPP (SEQ ID NO: 11) have a sequence identity of 6 out of 7 or 85%. In certain embodiments, any recitation of sequence identity expressed herein may be substituted for sequence similarity. Percent “similarity” is used to quantify the similarity between two sequences of the alignment. This method is identical to determining the identity except that certain amino acids do not have to be identical to have a match. Amino acids are classified as matches if they are among a group with similar properties according to the following amino acid groups: Aromatic—F Y W; hydrophobic—A V I L; Charged positive: R K H; Charged negative—D E; Polar—S T N Q.

As used herein a “growth medium” and “media” and “culture compositions” are used interchangeably to refers to a composition that contains components, such as vitamins, amino acids, inorganic salts, a buffer, and a fuel, e.g., acetate, succinate, and/or a saccharide, that support the growth and maintenance of cell lines. Components in the growth medium may be derived from blood serum or the growth medium may be serum-free. The growth medium may optionally be supplemented with albumin, lipids, insulin and/or zinc, transferrin or iron, selenium, ascorbic acid, and an antioxidant such as glutathione, 2-mercaptoethanol or 1-thioglycerol.

As used herein the term “allogeneic” with regard to comparing cells capable of and/or secreting antibodies and mesenchymal stromal/stem cells (MSCs) refers to cells that are genetically dissimilar because they are not derived from the same person, e.g., the antibody secreting cells and the mesenchymal stromal/stem cells (MSCs), which provide for proteins secreted from mesenchymal stromal/stem cells (MSCs) in a growth media, are not both derived from the same person. Cells derived from the same person are designated as “syngeneic.”

In certain embodiments, the disclosure contemplates a growth media disclosed herein having one or more of the following components the RPMI 1640 and R10 medium at, about, or greater than those provided in the tables herein. The term “about” refers to having more or less not exceeding 10, 20, 30, 40 or 50% by weight.

RPMI 1640 Medium contains the reducing agent glutathione and vitamins. RPMI 1640 Medium contains biotin, vitamin B12, and PABA. In addition, the vitamins inositol and choline are present. RPMI 1640 Medium does not contain substantial amounts of proteins, lipids, or growth factors. RPMI 1640 Medium is commonly supplemented with 1-5% or 5-10% Fetal Bovine Serum (FBS). RPMI 1640 Medium uses a sodium bicarbonate buffer system (2.0 g/L).

TABLE 1
Components RPMI 1640 Medium

| | Molecular | Concentration |
|---|---|---|
| | Weight | (mg/L) |
| Amino Acids | | |
| Glycine | 75.0 | 10.0 |
| L-Arginine | 174.0 | 200.0 |
| L-Asparagine | 132.0 | 50.0 |
| L-Aspartic acid | 133.0 | 20.0 |
| L-Cystine 2HCl | 313.0 | 65.0 |
| L-Glutamic Acid | 147.0 | 20.0 |
| L-Glutamine | 146.0 | 300.0 |
| L-Histidine | 155.0 | 15.0 |
| L-Hydroxyproline | 131.0 | 20.0 |
| L-Isoleucine | 131.0 | 50.0 |
| L-Leucine | 131.0 | 50.0 |
| L-Lysine hydrochloride | 183.0 | 40.0 |
| L-Methionine | 149.0 | 15.0 |
| L-Phenylalanine | 165.0 | 15.0 |
| L-Proline | 115.0 | 20.0 |
| L-Serine | 105.0 | 30.0 |
| L-Threonine | 119.0 | 20.0 |
| L-Tryptophan | 204.0 | 5.0 |
| L-Tyrosine disodium salt dihydrate | 261.0 | 29.0 |
| L-Valine | 117.0 | 20.0 |
| Vitamins | | |
| Biotin | 244.0 | 0.2 |
| Choline chloride | 140.0 | 3.0 |
| D-Calcium pantothenate | 477.0 | 0.25 |
| Folic Acid | 441.0 | 1.0 |
| Niacinamide | 122.0 | 1.0 |
| Para-Aminobenzoic Acid | 137.0 | 1.0 |
| Pyridoxine hydrochloride | 206.0 | 1.0 |
| Riboflavin | 376.0 | 0.2 |
| Thiamine hydrochloride | 337.0 | 1.0 |
| Vitamin B₁₂ | 1355.0 | 0.005 |
| i-Inositol | 180.0 | 35.0 |
| Inorganic Salts | | |
| Calcium nitrate (Ca(NO₃)₂ 4H₂O) | 236.0 | 100.0 |
| Magnesium Sulfate (MgSO₄) (anhyd.) | 120.0 | 48.84 |
| Potassium Chloride (KCl) | 75.0 | 400.0 |
| Sodium Bicarbonate (NaHCO₃) | 84.0 | 2000.0 |
| Sodium Chloride (NaCl) | 58.0 | 6000.0 |
| Sodium Phosphate dibasic | 142.0 | 800.0 |
| (Na₂HPO₄) anhydrous | | |
| Other Components | | |
| D-Glucose (Dextrose) | 180.0 | 2000.0 |
| Glutathione (reduced) | 307.0 | 1.0 |
| Phenol Red | 376.4 | 5.0 |

R10 medium

500 mL, RPMI 1640 medium
55 mL Heat-inactivated fetal calf serum (FCS)
5 mL, L-glutamine (200 mM solution)
5 mL, Penicillin/streptomycin (10,000 U per mL and 10 mg per mL)
5 mL 1M HEPES buffer

Other contemplated components in these growth medium include ascorbic acid, L-alanine, zinc sulfate, human transferrin, albumin, insulin, ammonium metavanadate, cupric sulfate, manganous chloride, sodium selenite, ethanolamine, and sodium pyruvate.

The term “saccharide” refers to multi-hydroxylated hydrocarbons which predominantly form one or more cyclic five and/or six membered nonaromatic oxygen containing cyclic isomers in aqueous solutions. The term includes monosaccharides, disaccharides, or polysaccharides such as glucose, dextrose, fructose, lactose, mannose, sorbitol, or sucrose.

The term, “irradiation” of the cells, refers to exposing the cells to a γ-irradiation source. In certain embodiments, one irradiates the cells at about or more than or 50, 60, 70, 75 or 76.6 rad/minute (e.g. ˜0.766 Gy/minute) and do so for at or more than 20, 25, 30, 35 or 40 minutes. ˜3,064 rad, or ˜30.64 Gy).

Factors of the Bone Marrow Microniche that Support Human Plasma Cell Survival and Immunoglobulin Secretion

Human antibody secreting cells (ASC) from the blood are found during vaccination or infection but rapidly apoptose unless they migrate to the bone marrow (BM). Yet, the elements of the BM microniche that are required to sustain plasma cells remain elusive. Here unique factors of the BM microniche are identified that maintain ASC from the blood for over 60 days in vitro. The components of the cell-free in vitro BM mimic consist of products from primary BM mesenchymal stromal cells (MSC), APRIL, and hypoxic conditions that are involved in the long-lived plasma cell (LLPC) maturation process. Comparative analysis of protein-protein interactions between BM MSC proteomics with differential RNA transcriptomics of the blood ASC and BM LLPC identified two major survival factors, fibronectin and YWHAZ. These BM MSC secretome proteins and hypoxic conditions play a role in LLPC survival utilizing mechanisms that downregulate mTORC1 signaling and upregulate hypoxia signatures. Elements of the BM survival niche (BM MSC secretome, APRIL, and hypoxic conditions) are important in maturation of blood ASC to BM LLPC. The proteins, fibronectin & YWHAZ, in the secretome, along with APRIL & specialized conditions (hypoxia) from the BM microniche that play a role in LLPC maturation process of survival.

In addition to local stromal cells within the bone marrow (BM) microniche, other cell types such as eosinophils, megakaryocytes, basophils, monocytes, and dendritic cells potentially playing a role in ASC survival. Additionally, specific signals include ligands for the receptor BCMA (APRIL (a proliferation-inducing ligand) and possibly BAFF (a B cell-activation factor). In mice, Mcl-1, which is an important negative regulator of apoptosis through BCMA signaling, was found to be essential for BM plasma cell survival. Other cytokines and chemokines have also been implicated such as IL-5, IL-6, TNFalpha, CXCL12 and signals acting through CD44. However, individual factors only partially support ASC for days 5 and are not sufficient for long-term survival.

Another unique characteristic of the BM microenvironment is its lower oxygen tension. Relative to other organs, the BM is naturally hypoxic with an O₂ tension <10 mmHg. However, whether hypoxia is beneficial, detrimental, or even involved in the maintenance of ASC requires further elucidation.

Unique features characteristic of the BM microniche were tested to evaluate their contribution to the survival of primary human blood ASC in culture. Using IgG Elispots to measure individual plasma cell survival and function, cellular cocultures, cell-free secretomes of BM MSC were evaluated alone as well as in combination with exogenous cytokines under normoxic and hypoxic conditions.

This approach lead to an in vitro system able to sustain human ASC for several months. In addition, an integrated genomic approach was used to match potential protein-protein interactions identified from the MSC secretory proteome with genes that were differentially expressed between circulating human ASC and LLPC. This analysis identified new proteins, fibronectin and YWHAZ, in the MSC secretome along with APRIL and specialized conditions (hypoxia) from the BM microniche that play a role in LLPC maturation process of survival.

### Examples

### In Vitro Culture Systems for Human Blood and BM ASC.

BM-MSCs as feeder (cocultures on adherent monolayer) were co-cultured in 96-well flat-bottom cell culture plates or transwells (0.4 μm pore polycarbonate insert membrane of 96-well plates) in 37° C. in a humid, 5% CO₂, 95% air (20% O₂) incubator or in hypoxic culture conditions (2.5% (2) at 37° C. in a modular incubator chamber that was infused with a pre-analyzed gas mixture containing 2.5% O₂, 5% CO₂, and 92.5% N₂ (AirGas) or a cell culture incubator programmed for the desired O₂ tension. The input ASC numbers per well varied (˜100 to ˜3,082 cells per well) depending upon post-sort cell counts. In MSC secretome media, the same number of ASC alone were cultured with factors for specified days. For MSC-free cultures or conventional media, RPMI with 10% fetal bovine serum (R10) were used. Cells were harvested on designated days. The blood or BM ASC survival and function were assessed by Elispot assays, and their output values were expressed as the percentage of maximal IgG secreting ASC which typically occurred on days 1-3. Exogenous factors included a variety of cytokines and growth factors, IL-5, IL-6, APRIL, BAFF, IFNg, IL-21, bFGF, and CXCL12 which were added to cultures at day 0 with ASC.

### MSC Co-Cultures Support Short-Term ASC Survival.

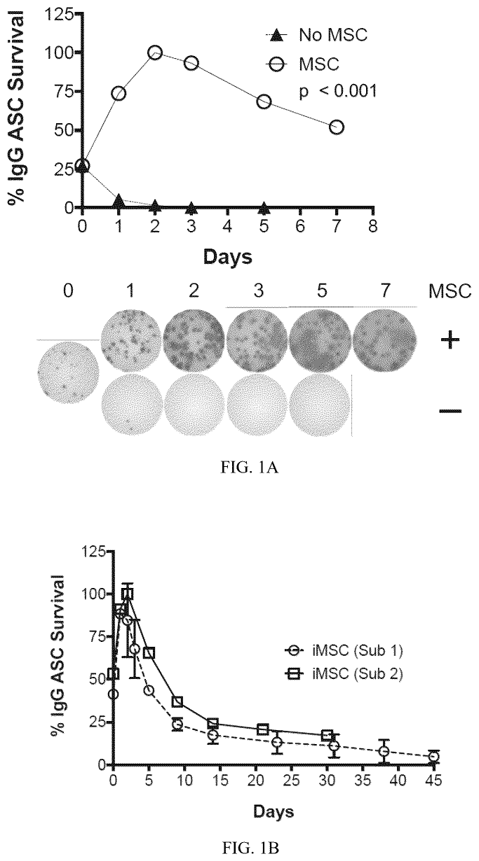

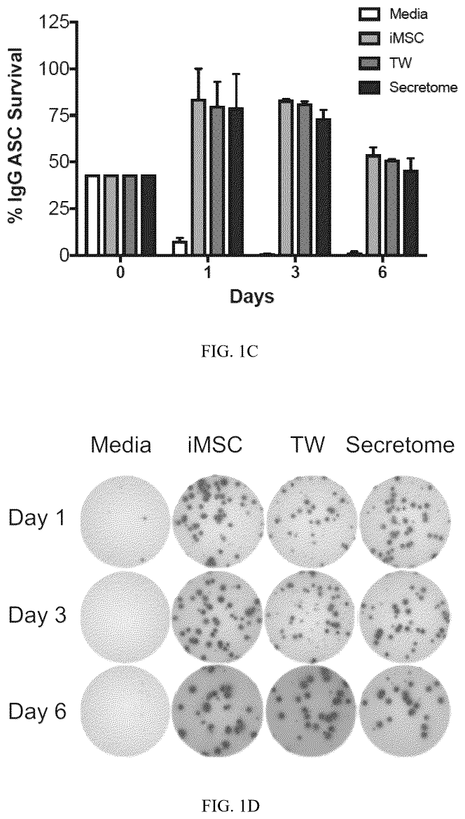

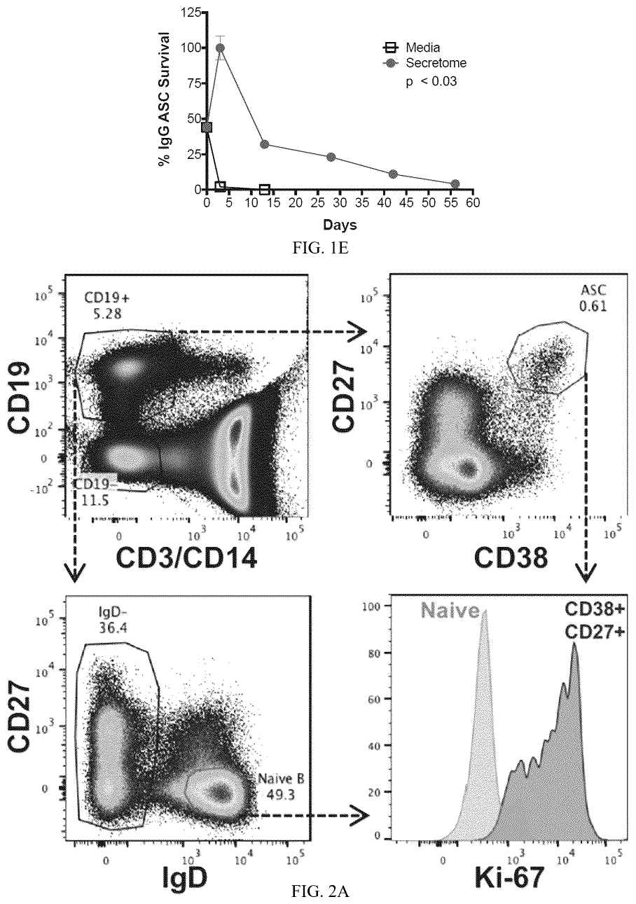

Circulating ASC were FACS purified (identified by CD19+CD38hiCD27hi) from two healthy adults, one at steady state and another 7 days after vaccination with PNEUMOVAX®23 (PPSV23). Sorted cells (1,000-1,500 ASC/well) were cultured in conventional media (RPMI with 10% fetal bovine serum (FBS)) or co-cultured with human BM-derived irradiated MSC (iMSC) for 7 days. Elispots were performed daily for 7 days to enumerate IgG secreting cells. Consistent with the pronounced death rate of human ASC ex vivo, very few ASC could be detected on day one and were essentially absent by day 3 when cultured in conventional media (FIG. 1A). In contrast, >50% of the maximal ASC seeded in MSC co-cultures readily survived for 7 days. Maximal IgG ASC were determined by the peak of the Elispot responses, which typically occurred on days 1-3 of the cultures.

### Long-Term Survival and Function of ASC in MSC Co-Cultures.

The ability of iMSC to support ASC survival in culture for extended periods of time was evaluated. iMSC rather than non-irradiated MSC (noniMSC) were used for these studies to avoid MSC outgrowth, senescence, and possible consumption of essential nutrients required for ASC. Two ASC samples (one from a healthy adult at steady state and another 8 days after Tetanus, Diphtheria, Pertussis (Tdap) vaccination) were cultured at 333-1,500 ASC/well with 25,000 or 30,000 iMSC/well for up to 45 days. The co-cultures were tested by IgG Elispot daily for the first 3 days and weekly thereafter. As before, a significant increase in the frequency of IgG ASCs were detected within the first 72 hours of culture. Approximately 20-25% of the maximal IgG ASC secreted IgG after 2 weeks in culture and 15-20% after 31 days. While this represents an improvement relative to cultures devoid of iMSC, this drop in survival suggested that replenishment of iMSC or additional factors are needed to sustain long-lived survival of ASC.

### MSC Support ASC Survival is not Dependent on Cell-Cell Contact.

To understand if cell-cell contact is needed for plasma cell survival, 1,000-2,500 blood ASC obtained from two subjects after hepatitis B vaccination and 2014-2015 trivalent influenza vaccination (TIV) were co-cultured directly on iMSC, with iMSC in transwells (0.4 μm pore size), or with MSC secretome alone. No significant differences in IgG Elispot frequencies were observed between direct MSC: ASC co-cultures or transwell MSC: ASC co-cultures at any time point within 6 days; thereby, establishing that cell-cell contact is not necessary for ASC survival (FIG. 1C, 1D). As before, ASC in media alone died within one day, but a significant recovery of ASC function was observed within 24 hours of co-cultures. Whether secreted MSC factors were enough to support ASC survival was tested. To do so, soluble secreted factors were isolated from MSC cultures and it was determined that, in isolation, the MSC secretome sustained ASC survival equal to cocultures and transwells for 6-7 days (p-value<0.925).

Similar to the long-term co-cultures with iMSC, the ability of the MSC secretome to support ASC survival and IgG secretion could be extended in culture for up to 56 days (FIG. 1E). However, also similar to the co-cultures with iMSC, only 20% of the ASC survived by day 30 suggesting additional factors are needed to sustain ASC long-term.

### MSC Secretomes Restore the Secretory Function of Sorted ASC.

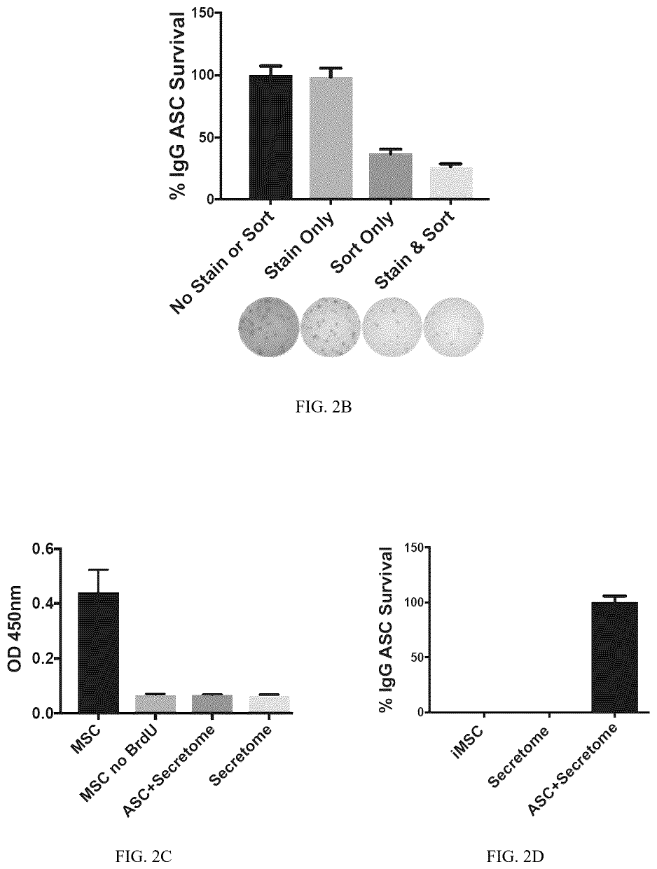

As shown in FIG. 1A, 1B, IgG Elispot frequencies of co-cultured ASC increased greatly during the first 72 hours of culture compared to day 0 (representative of over 10 experiments). Nearly all input ASC from the blood 7 days post-vaccination were Ki67+ 1 (FIG. 2A) suggesting recent or ongoing proliferation of ASC. Thus, the increase in IgG ASC early in the cultures could be explained either by in vitro proliferation or restoration of IgG secretory function from non-proliferating (but recently proliferated) ASC. Because the decreased functionality immediately after sorting could be mediated by the high pressure stress of FAC sorting or the staining process, Elispots of equal numbers of peripheral blood mononuclear cells (PBMC) were compared after staining with the antibody panel (including CD27 and CD38) or leaving them untouched (no staining). In isolation, this manipulation did not decrease the overall spot numbers (FIG. 2B). However, the impact of FACS sorting, which creates significant sheer stress, caused a nearly 3-fold reduction in number of IgG Elispots compared to untouched PBMC despite the same number of total PBMC that were collected after being subjected to the high pressure FACS. The greatest reduction in functional IgG ASC was observed with a combination of staining and sorting on PBMC (p<0.002).

To assess for ASC proliferation in the cultures, BrdU was added to the wells with secretome alone or secretome with ASC from 2 different subjects for 2 days. As positive controls, noniMSC, which actively proliferate in culture, showed a positive OD450 value of 0.44. As negative controls, the OD450 of <0.1 was measured in MSC without BrdU added (FIG. 2C). When tested, the BM MSC secretome alone was negative for BrdU uptake which was not surprising since filtering through a 0.2 μm filter would have removed any remaining cells. However, surprisingly, the ASC incubated with the secretome was also negative for BrdU uptake demonstrating that the human ASC found in circulation after vaccination are not actively dividing in the cultures despite positive Ki67 staining showing cells have undergone recent proliferation but are not actively proliferating (p<0.004). ASC survival and IgG secretory function are diminished by FACS sorting and recover from a stunned non-secretory state without further proliferate within 48 h in the culture conditions.

To ensure that the iMSC or the secretome did not contain contaminating plasma cells derived from the BM, IgG Elispots from the iMSC cultures and their secretome alone were performed. No IgG Elispots were detected in the iMSC alone or the MSC secretome cultures (FIG. 2D). IgG ASC in the cultures were only detected with the secretome together with sorted blood ASC demonstrating IgG Elispots originated from input blood ASC and not from any contaminating BM plasma cells carried over with the iMSC or the MSC secretome preparations.

Since ASC were not proliferating in the cultures and the numbers of viable IgG ASC varied with each sample and FAC sort, we normalized each experiment to the maximal IgG Elispot numbers detected on days 1-3. On day 0, approximately 3-30% of input cells were able to secrete IgG antibodies. This number may be low due to several reasons: overestimates of cell counts after FAC sorting, apoptosis due to sheer stress of cells after collection, or loss with washing steps. Additionally, from blood CD19loCD27hiCD38hi sorted cells, a significant percentage of IgA circulating ASC can account for nonIgG secretors that could range from 10-60%. In order to study the long-term survival and function of ASC in BM maturation conditions, percentage of subsequent ASC survival beyond day 1 was measured.

### APRIL Enhances ASC Survival Induced by the MSC Secretome.

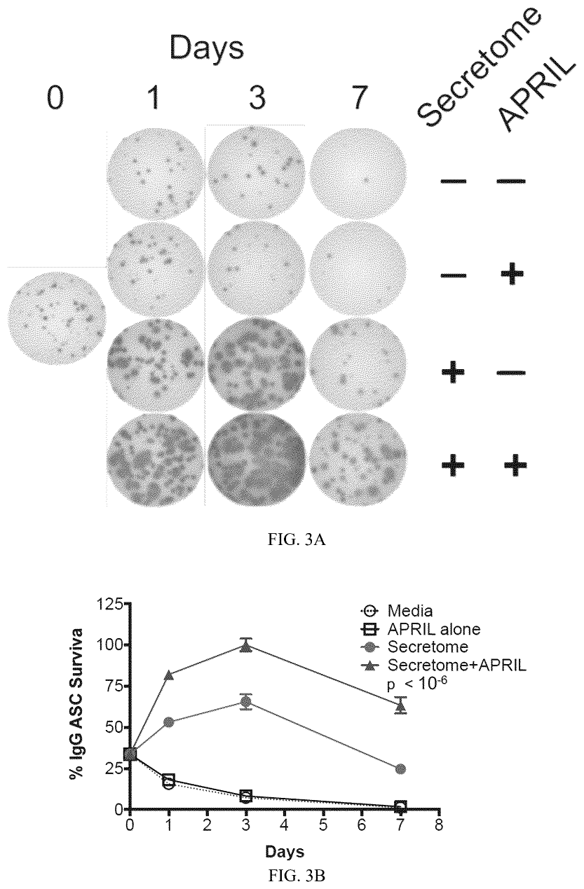

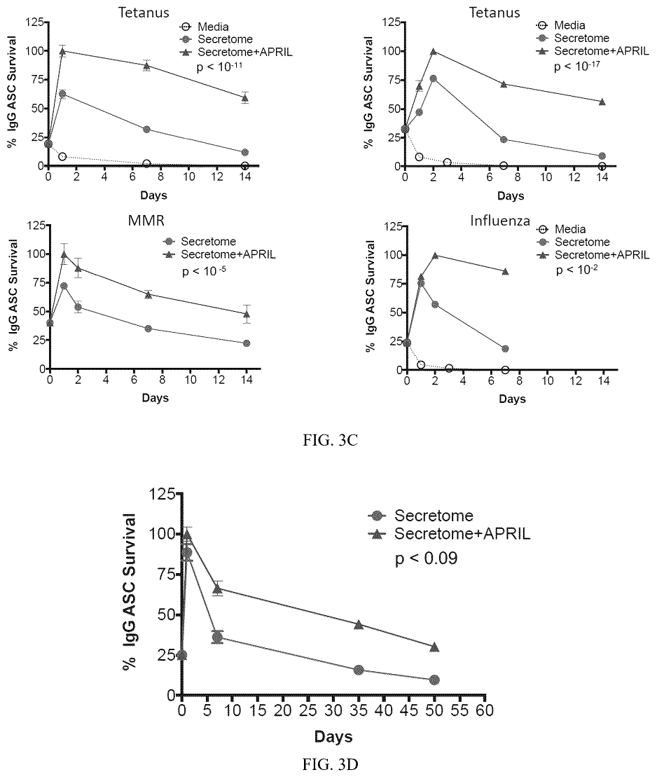

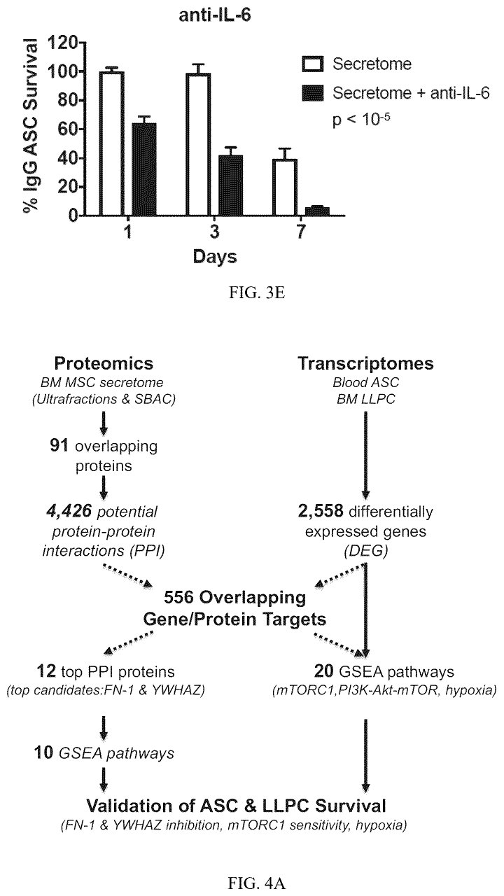

B cell maturation antigen (BMCA), a TNF superfamily (TNFRS17) receptor for APRIL, is highly expressed by all human BM ASC including the LLPC. Thus, the effect of APRIL alone or in combination with the MSC secretome on the survival of ASC was tested. As shown in FIGS. 3A and 3B, exogenous APRIL alone provided no ASC survival advantage over conventional media. A similar lack of benefit was observed with other individual cytokines such as IL-5, IL-6, and BAFF alone.

Despite APRIL providing no survival advantage when used in isolation, the addition of exogenous APRIL to the MSC secretome resulted in more than 2-fold enhancement in functional IgG ASC frequency over the first 24 hours of culture followed by significantly enhanced ASC survival over 7 days (p<10-6) (FIGS. 3A and 3B). This increase was significantly greater than ASC cultured with the MSC secretome alone from days 1 to 7. This phenomenon was statistically significant in four additional subjects after vaccines including tetanus, influenza virus, and measles & mumps-rubella (MMR) (FIG. 3C). Interestingly, the addition of APRIL to the MSC secretome promoted long-lasting ASC survival (14 days and beyond) at rates significantly superior to the activity of the MSC secretome alone. This additional survival benefit of exogenous APRIL was sustained up to 56 days which trended towards significance (p value<0.09) (FIG. 3D). Thus, the presence of APRIL in the MSC secretome enhanced short-term (days) and likely long term (weeks) ASC survival.

In contrast to APRIL, the addition of BAFF to the MSC secretome provided no additional survival benefit. Furthermore, the addition of IL-6, IL-5, IFNg, VEGF, or basic Fibroblast Growth Factor (bFGF or FGF2) to the MSC secretome also offered no further enhancement of survival. Despite additional IL-6 conferring no benefit, inhibition with IL-6 antibodies in the secretome significantly diminished survival (FIG. 3e) on days 1, 3, and 7 compared to MSC secretome alone (p<10-5) demonstrating that the secretome contained abundant amounts of IL-6.

### Proteomics of the MSC Secretome.

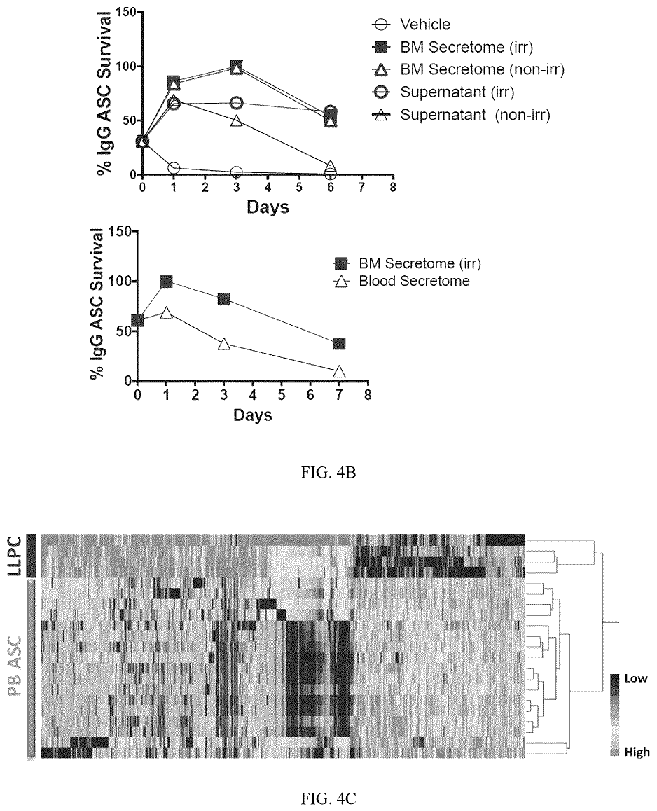

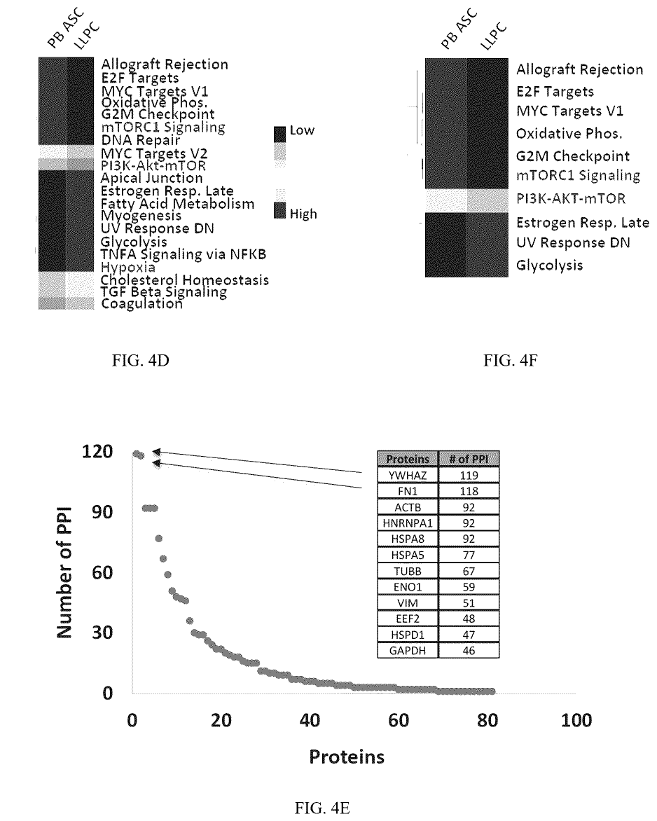

To identify novel MSC plasma cell survival factors a combined bioinfomatic approach of proteomics and transcriptomics were utilized (FIG. 4A). Initially, proteomics of MSC secretome fractions was performed with and without ASC survival activity. Secretomes were isolated from irradiated and non-irradiated MSC and fractionated the supernatants by ultracentrifugation. Additionally, secretomes from blood (not BM) adherent cells (SBAC) were also generated. The fractions with biological activity included the irradiated or non-irradiated MSC secretomes and the irradiated supernatant, whereas, the non-irradiated supernatant and the SBAC showed decreased ASC survival (FIG. 4B). Proteomics was performed on irradiated and non-irradiated secretomes and irradiated and non-irradiated supernatants along with the SBAC fraction, and peptide fragments were aligned using DAVID. Background of fetal bovine serum was subtracted. The union of the positive biologic fractions (irradiated and non-irradiated secretomes together with the irradiated supernatant) yielded 231 proteins. One hundred fifty-six proteins were identified from the irradiated MSC secretome proteome subtracting the SBAC proteome. The number of overlapping proteins between the two experiments was 91. Then, HIPPIE was employed to discover 4,426 potential protein-protein interactions (PPI) with the 91 MSC proteins.

To further narrow the MSC PPI that may play a role in ASC survival process, the differentially expressed genes (DEG) between the early minted ASC in the blood was compared to the LLPC in the BM (SRA: SRP057017). Transcriptomes of 17 new blood ASC samples using the same surface markers described in BM ASC subsets (pop A, B, and pop D (LLPC)) 1 and the 4 BM LLPC (pop D) were aligned, normalized, and analyzed with standard statistical methods. A total of 2,558 genes were found to be differentially expressed between blood ASC and BM LLPC at an FDR of 0.05 (FIG. 4C). Using an integrated approach designed to assess the role of the MSC proteome on the LLPC maturation, the 4,426 potential PPI from the MSC proteome were overlapped with the 2,558 DEG between blood ASC and BM LLPC transcriptomes and identified 556 overlapping gene/protein targets. Gene set enrichment analysis (GSEA) revealed 20 statistically significant Hallmark pathways (FIG. 4D)). Highlighted pathways included mTORC1 signaling, PI3K-AktmTOR signaling, TNFalpha signaling, and hypoxia.

Twelve of the 91 proteins that had more than 45 PPI that overlapped with the DEG profiles of blood ASC to BM LLPC (FIG. 4E). The top proteins consisted of fibronectin (FN-1), YWHAZ (also known as 14-3-3 zeta/delta), heat shock proteins, endolase, glyceraldehyde 3-phosphate dehydrogenase (GAPDH), eukaryotic elongation factor (EEF2), and various cytoskeletal proteins (actin, tubulin, & vimentin). The top two proteins, FN-1 and YWHAZ, had 436 and 430 PPI, respectively, of which 118 and 119 overlapped with the DEG in blood ASC and LLPC (FDR<0.05; p-value<0.05). The pathways involved for only FN-1 and YWHAZ involved in only 10 of the 20 GSEA hallmark pathways (FIG. 4F)). These pathways included proliferation signatures i.e. E2F targets and G2M checkpoints, which is consistent with evidence that most blood ASC have undergone recent proliferation (FIG. 2A) in contrast to the BM LLPC 1. However, the downregulation of Myc targets, mTORC1 signaling, and PI3K-Akt-MTOR signaling suggested novel pathways that the MSC secretome may be involved in maintaining ASC survival.

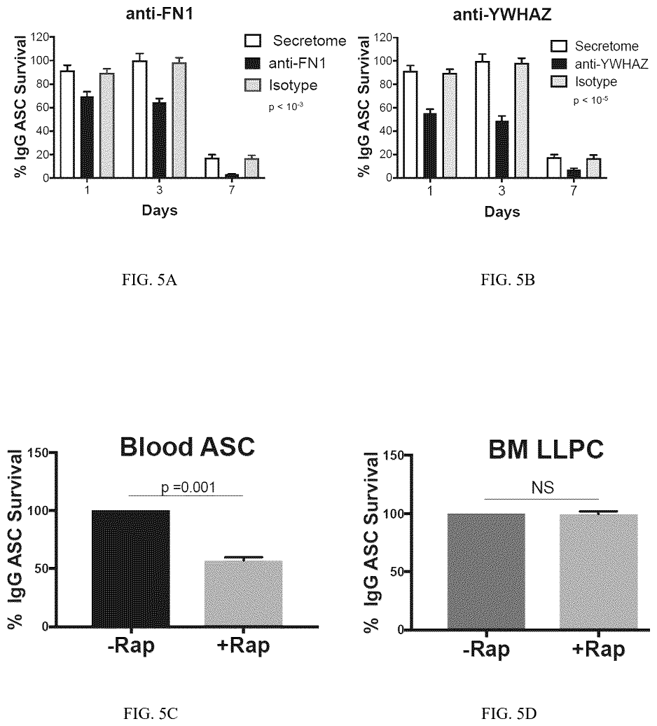

To validate the importance of the top two proteins, FN-1 and YWHAZ were inhibited with anti-fibronectin and anti-YWHAZ antibodies in the MSC secretome and showed statistically significant decrease in ASC survival after 1, 3, and 7 days compared to no inhibition or isotype controls (p<10-3 and <10-5) (FIGS. 5A and 5B). Unique potential proteins were identified from the MSC secretome that may play a role in ASC survival. FN-1 and YWHAZ had the strongest PPI and their inhibition resulted in decreased ASC survival.

Role of mTORC1 Signaling in LLPC Maturation.

Integrated proteomics and transcriptomics posited a role for the MSC secretome in downregulating mTORC1 signaling as early blood ASC enter the BM microniche and mature into LLPC. It may be that early-minted blood ASC would be sensitive to rapamycin, an mTORC1 inhibitor, while BM LLPC would be resistant to rapamycin. Indeed, blood ASC showed decreased survival with rapamycin validating enhanced mTORC1 signaling in the blood ASC from three subjects (FIG. 5C). In contrast, BM LLPC from three adult BM samples were entirely resistant to rapamycin confirming the downregulation of mTORC1 signaling in LLPC (FIG. 5D). These results highlight the importance of proteins from the MSC secretome such as FN-1 and YWHAZ and their putative role in downregulating mTORC1signaling as early-minted blood ASC mature into LLPC.

### ASC Survival is Enhanced Under Hypoxic Conditions.

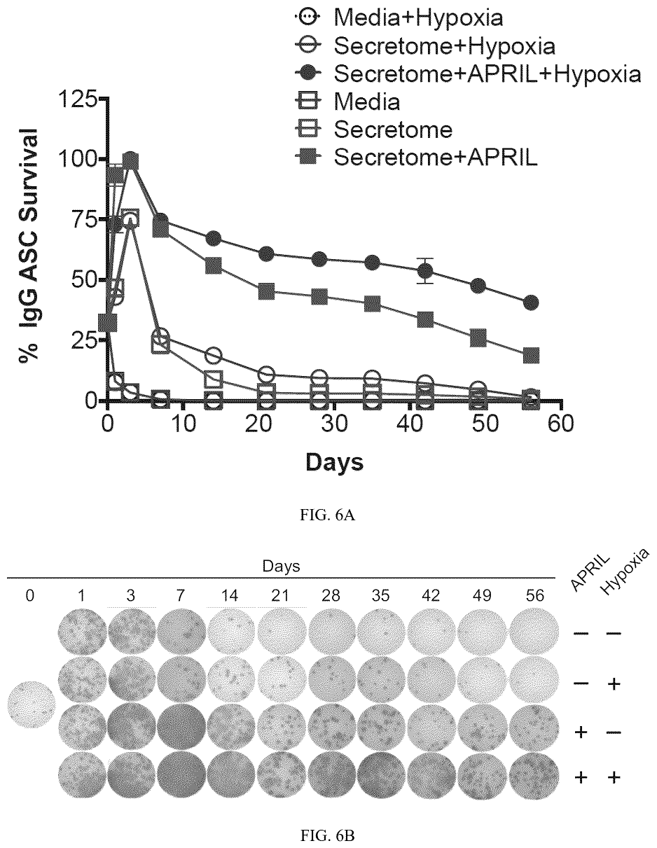

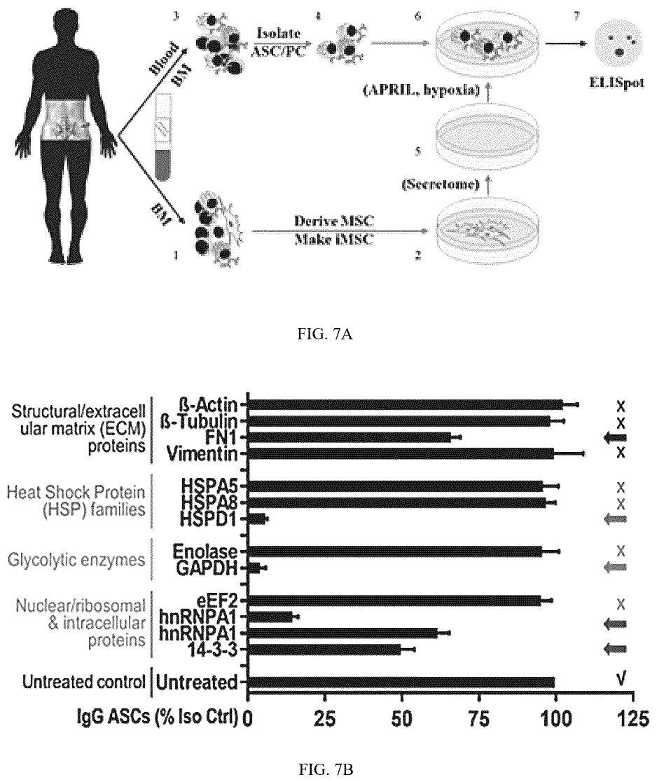

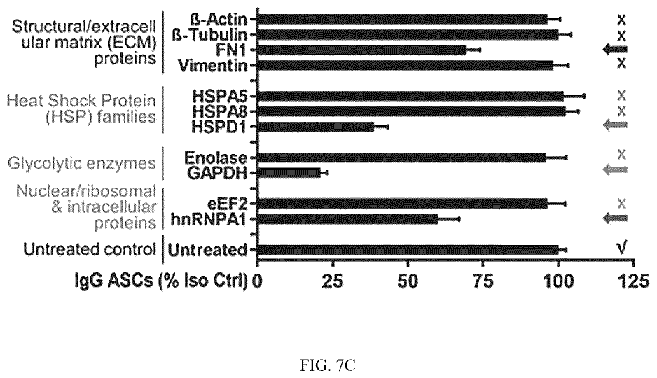

Of the 20 GSEA hallmark pathways discovered with the integrated bioinformatics of the MSC proteome and DEG of the blood-BM transcriptomes, hypoxia hallmark pathways were prominently featured. Interestingly, the hypoxia pathway is not enriched for interactions involving FN-1 and YWHAZ suggesting that upregulation of hypoxia signatures are independent of these top proteins (FIGS. 4C and 4D). To investigate the role of hypoxia in ASC survival, circulating ASCs were isolated 7 days after tetanus immunization and ASCs survival were measured in the following three conditions in normoxia and hypoxia: conventional media alone, MSC secretome, and MSC secretome with APRIL (FIGS. 6A and 6B). In keeping with the previously described results (FIGS. 1B and 1E), conventional media in normoxic or hypoxic conditions provided no support.

The MSC secretome alone under normoxia showed only moderate ASC survival (9%±1%) by day 14, and under hypoxia, the same conditions improved ASC survival at day 14 (18%±2%). The MSC secretome with APRIL improved survival particularly beyond 14 to 56 days in normoxic conditions (56% and 19%, respectively). However, hypoxic conditions in combination of APRIL and the MSC secretome further enhanced ASC survival to over 66% at day 14 and over 40% at day 56. Thus, in this context, hypoxia significantly enhanced ASC survival in the MSC secretome and APRIL combined at every time point from day 14 to 56 (p-value 10-26). Together, these results demonstrate the prominent synergistic effect on ASC survival with hypoxia in combination with the MSC secretome and APRIL, suggesting that BM environmental factors play significant roles in novel mechanisms of LLPC maturation leading to prolonged survival.

Protein Factors Such as Fibronectin, YWHAZ, GAPDH, HSPD1, and hnRNPA1 are Important for Human ASC/PC Survival.

Blood ASC were cultured in secretome without and with treatment with individual specific Ab or small molecule inhibitors targeting GAPDH, HSPD1, or hnRNPA1, all of which were used at their half maximal inhibitory concentration at the beginning of cultures. These experiments, indicate multiple factors in the MSC secretome that are important for the maintenance and survival of ASC. In addition to IL-6, fibronectin, and APRIL, these factors include YWHAZ, HSPD1, GAPDH, and HNRNPA1. Specific blockade or inhibition of GAPDH, HSPD1, or hnRNPA1 resulted in loss of human ASC/PC IgG ELISpot frequencies. To understand whether ASC/PC secretion or survival was affected, the inhibitors were sequentially removed and re-plated in the previously-treated ASC/PC in fresh conditions. Upon removal of the inhibitors, the inhibitory effects of Ab secretion were reversed, suggesting a functional recovery of Ab secretion of the inhibited ASC/PC. No evidence of additional ASC/PC proliferation was observed by BrdU uptake. Given the physiological functions of GAPDH, HSPD1, and hnRNPA1, it appeared that mechanisms involved in glucose or mitochondrial metabolism, or protein synthesis, can modulate Ab secretion of human ASC/PC.

What is claimed is:

1. A cell culture composition comprising exogenously added tyrosine 3-monooxygenase/tryptophan 5-monooxygenase activation protein zeta protein (YWHAZ), IL-6, and fibronectin.

2. The cell culture composition of claim 1, wherein the composition further comprises exogenously added A-proliferation-inducing ligand (APRIL).

3. The cell culture composition of claim 1, wherein the composition further comprises exogenously added Beta-actin (ACTB), Heterogeneous nuclear ribonucleoprotein A1 (HNRNPA1), Heat shock 70 kDa protein 8 (HSPA8), heat shock 70 kDa protein 5 (HSPA5), Tubulin beta chain (TUBB), alpha-enolase (ENO1), Vimentin (VIM), Eukaryotic elongation factor 2 (EEF2), heat shock protein family D (Hsp60) member 1 (HSPD1), Glyceraldehyde 3-phosphate dehydrogenase (GAPDH), or combinations thereof.

4. The cell culture composition of claim 1, wherein the composition further comprises secretions of allogeneic mesenchymal stromal/stem cells.

5. The cell culture composition of claim 1 in an enclosure wherein the amount of oxygen is less than 5% by volume.

6. The cell culture of claim 1 further comprising isolated cells capable of producing antibodies.

7. The cell culture composition of claim 1 further comprising exogenously added components selected from buffering agent, amino acids and vitamins.

8. A method of culturing cells that are capable of producing antibodies comprising mixing cells capable of producing antibody with a cell growth medium of claim 1.

9. The method of claim 8, wherein cells that are capable of producing antibodies are plasma cells or antibody-secreting cells (ASCs).

10. The method of claim 9, wherein the plasma cells have surface molecules in a pattern wherein no or low levels of CD19 are expressed, CD138 is expressed, and CD38 is expressed in higher levels than CD138.

11. A method of sequencing a nucleic acid of antibodies that specifically bind to an antigen comprising
isolating antibody secreting cells from a sample, providing separated single antibody secreting cells in a plurality of separate areas;
contacting the separated single antibody secreting cells in the plurality of separated areas with a culture composition comprising exogenously added YWHAZ, IL-6, and fibronectin under conditions such that separated single antibody secreting cells replicate providing replicated homogenous antibody secreting cells in separate areas;
identifying replicated homogenous antibody secreting cells that produce antibodies that specifically bind to an antigen; and
sequencing a nucleic acid that encodes the antibody in the replicated homogenous antibody secreting cells that bind the antigen.

12. The method of claim 11, wherein the culture composition further comprises exogenously added A-proliferation-inducing ligand (APRIL), fragment, or variant thereof.

13. The method of claim 11, wherein the culture composition further comprises exogenously added Beta-actin (ACTB), Heterogeneous nuclear ribonucleoprotein A1 (HNRNPA1), Heat shock 70 kDa protein 8 (HSPA8), heat shock 70 kDa protein 5 (HSPA5), Tubulin beta chain (TUBB), alpha-enolase (ENO1), Vimentin (VIM), Eukaryotic elongation factor 2 (EEF2), heat shock protein family D (Hsp60) member 1 (HSPD1), Glyceraldehyde 3-phosphate dehydrogenase (GAPDH), or combinations thereof.

14. The method of claim 11, wherein the culture composition further comprises secretions of allogeneic mesenchymal stromal/stem cells and

15. The method of claim 11, wherein the culture composition is in an enclosure wherein the amount of oxygen is less than 5% by volume.

16. The method of claim 11 wherein the sample is blood, bone marrow, or product derived therefrom.

17. The method of claim 11 wherein isolating antibody secreting cells is accomplished by fluorescence activated cell sorting.


SEQUENCE LISTING (11 sequences)

SEQ ID NO: 1 - protein, 250 aa, Artificial [sequence omitted]

SEQ ID NO: 2 - protein, 212 aa, Artificial
  Synthetic construct
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 3 - protein, 135 aa, Artificial
  Synthetic construct
CESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 4 - protein, 245 aa, Artificial
  Synthetic construct
MDKNELVQKAKLAEQAERYDDMAACMKSVTEQGAELSNEERNLLSVAYKNVVGARRSSWRVVSSIEQKTEGAEKKQQMAREYREKIETELRDICNDVLSLLEKFLIPNASQAESKVFYLKMKGDYYRYLAEVAAGDDKKGIVDQSQQAYQEAFEISKKEMQPTHPIRLGLALNFSVFYYEILNSPEKACSLAKTAFDEAIAELDTLSEESYKDSTLIMQLLRDNLTLWTSDTQGDEAEAGEGGEN

SEQ ID NO: 5 - protein, 573 aa, Artificial
  Synthetic construct
MLRLPTVFRQMRPVSRVLAPHLTRAYAKDVKFGADARALMLQGVDLLADAVAVTMGPKGRTVIIEQSWGSPKVTKDGVTVAKSIDLKDKYKNIGAKLVQDVANNTNEEAGDGTTTATVLARSIAKEGFEKISKGANPVEIRRGVMLAVDAVIAELKKQSKPVTTPEEIAQVATISANGDKEIGNIISDAMKKVGRKGVITVKDGKTLNDELEIIEGMKFDRGYISPYFINTSKGQKCEFQDAYVLLSEKKISSIQSIVPALEIANAHRKPLVIIAEDVDGEALSTLVLNRLKVGLQVVAVKAPGFGDNRKNQLKDMAIATGGAVFGEEGLTLNLEDVQPHDLGKVGEVIVTKDDAMLLKGKGDKAQIEKRIQEIIEQLDVTTSEYEKEKLNERLAKLSDGVAVLKVGGTSDVEVNEKKDRVTDALNATRAAVEEGIVLGGGCALLRCIPALDSLTPANEDQKIGIEIIKRTLKIPAMTIAKNAGVEGSLIVEKIMQSSSEVGYDAMAGDFVNMVEKGIIDPTKVVRTALLDAAGVASLLTTAEVVVTEIPKEEKDPGMGAMGGMGGGMGGGMF

SEQ ID NO: 6 - protein, 293 aa, Artificial
  Synthetic construct
MVYMFQYDSTHGKFHGTVKAENGKLVINGNPITIFQERDPSKIKWGDAGAEYVVESTGVFTTMEKAGAHLQGGAKRVIISAPSADAPMFVMGVNHEKYDNSLKIISNASCTTNCLAPLAKVIHDNFGIVEGLMTTVHAITATQKTVDGPSGKLWRDGRGALQNIIPASTGAAKAVGKVIPELNGKLTGMAFRVPTANVSVVDLTCRLEKPAKYDDIKKVVKQASEGPLKGILGYTEHQVVSSDFNSDTHSSTFDAGAGIALNDHFVKLISWYDNEFGYSNRVVDLMAHMASKE

SEQ ID NO: 7 - protein, 320 aa, Artificial
  Synthetic construct
MSKSESPKEPEQLRKLFIGGLSFETTDESLRSHFEQWGTLTDCVVMRDPNTKRSRGFGFVTYATVEEVDAAMNARPHKVDGRVVEPKRAVSREDSQRPGAHLTVKKIFVGGIKEDTEEHHLRDYFEQYGKIEVIEIMTDRGSGKKRGFAFVTFDDHDSVDKIVIQKYHTVNGHNCEVRKALSKQEMASASSSQRGRSGSGNFGGGRGGGFGGNDNFGRGGNFSGRGGFGGSRGGGGYGGSGDGYNGFGNDGSNFGGGGSYNDFGNYNNQSSNFGPMKGGNFGGRSSGPYGGGGQYFAKPRNQGGYGGSSSSSSYGSGRRF

SEQ ID NO: 8 - protein, 6 aa, Artificial
  Synthetic construct
GGGGGG

SEQ ID NO: 9 - protein, 5 aa, Artificial
  Synthetic construct
GGGGT

SEQ ID NO: 10 - protein, 6 aa, Artificial
  Synthetic construct
GGGPPP

SEQ ID NO: 11 - protein, 7 aa, Artificial
  Synthetic construct
GGGAPPP